(19) United States
(12) Patent Application Publication
(10) Pub. No.: US 2018/0049627 A1
(43) Pub. Date: Feb. 22, 2018

(54) IMAGING UNIT AND ENDOSCOPE

(71) Applicant: OLYMPUS CORPORATION, Tokyo (JP)

(72) Inventors: Satoru ADACHI, Tsuchiura-shi (JP); Noriyuki FUJIMORI, Suwa-shi (JP); Takatoshi IGARASHI, Ina-shi (JP)

(73) Assignee: OLYMPUS CORPORATION, Tokyo (JP)

(21) Appl. No.: 15/794,039

(22) Filed: Oct. 26, 2017

Related U.S. Application Data
(63) Continuation of application No. PCT/JP2016/061735, filed on Apr. 11, 2016.

(30) Foreign Application Priority Data
Jun. 19, 2015 (JP) 2015-124066

(51) Int. Cl. A61B 1/05; G02B 23/24; G02B 23/26; H04N 5/30

(52) U.S. Cl. CPC A61B 1/05; G02B 23/2407; G03B 17/02; H04N 5/30; G02B 23/26

(57) ABSTRACT

An imaging unit includes: an image sensor configured to generate an image signal by receiving light and performing photoelectric conversion; and a relay member including a plurality of silicon substrates laminated on a back surface side of the image sensor opposite to a light receiving surface of the image sensor, planar type electronic devices being formed on the silicon substrates, and relay the image sensor and a signal cable that transmits the image signal. The relay member includes a multilayer wiring layer laminated on an outermost surface of the silicon substrate, and the multilayer wiring layer includes, on an outermost surface, a material allowing the signal cable to be connected.

### CROSS-REFERENCE TO RELATED APPLICATION

This application is a continuation of PCT international application Ser. No. PCT/JP2016/061735 filed on Apr. 11, 2016 which designates the United States, incorporated herein by reference, and which claims the benefit of priority from Japanese Patent Applications No. 2015-124066, filed on Jun. 19, 2015, incorporated herein by reference.

### BACKGROUND

The present disclosure relates to an imaging unit and an endoscope.

In the related art, an endoscope acquires an in-vivo image in a subject such as a patient by inserting, into the subject, a flexible insertion portion having an elongated shape provided with an imaging device at a distal end. An imaging unit used in such an endoscope includes a semiconductor chip on which an image sensor is formed, and a circuit board which is disposed adjacent to a back surface side of the semiconductor chip and on which electronic components such as a capacitor, a resistor, and an IC chip that constitute a driving circuit of the image sensor are mounted (see Japanese Patent No. 4575698 and Japanese Patent No. 4441305).

### SUMMARY

An imaging unit may include: an image sensor configured to generate an image signal by receiving light and performing photoelectric conversion; and a relay member including a plurality of silicon substrates laminated on a back surface side of the image sensor opposite to a light receiving surface of the image sensor, planar type electronic devices being formed on the silicon substrates, and relay the image sensor and a signal cable that transmits the image signal, the relay member includes a multilayer wiring layer laminated on an outermost surface of the silicon substrate, and the multilayer wiring layer includes, on an outermost surface, a material allowing the signal cable to be connected.

The above and other objects, features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating an overall configuration of an endoscope system according to a first embodiment;

FIG. 2 is a partial cross-sectional view of a distal end portion of an endoscope according to the first embodiment;

FIG. 3 is a cross-sectional view of an imaging unit according to the first embodiment;

FIG. 4 is a cross-sectional view of an imaging unit according to a second embodiment;

FIG. 5 is a cross-sectional view of a relay member according to a first modification of the embodiments;

FIG. 6 is a cross-sectional view of a relay member according to a second modification of the embodiments;

FIG. 7 is a schematic cross-sectional view of an imaging unit according to a third modification of the embodiments;

FIG. 8 is a schematic cross-sectional view of another imaging unit according to the third modification of the embodiments;

FIG. 9 is a schematic cross-sectional view of an imaging unit according to a fourth modification of the embodiments; and

FIG. 10 is a schematic cross-sectional view of another imaging unit according to the fourth modification of the embodiments.

### DETAILED DESCRIPTION

Hereinafter, an endoscope including an imaging device will be described as a mode for carrying out the present disclosure (hereinafter referred to as an “embodiment”). In addition, the present disclosure is not limited by the embodiment. Further, each drawing referred to in the following description only schematically illustrates a shape, a size, and a positional relationship to the extent that contents may be understood. That is, the present disclosure is not limited only to the shape, the size and the positional relationship exemplified in each drawing. Furthermore, dimensions and ratios may be differently illustrated among the drawings.

### First Embodiment

Configuration of Endoscope System

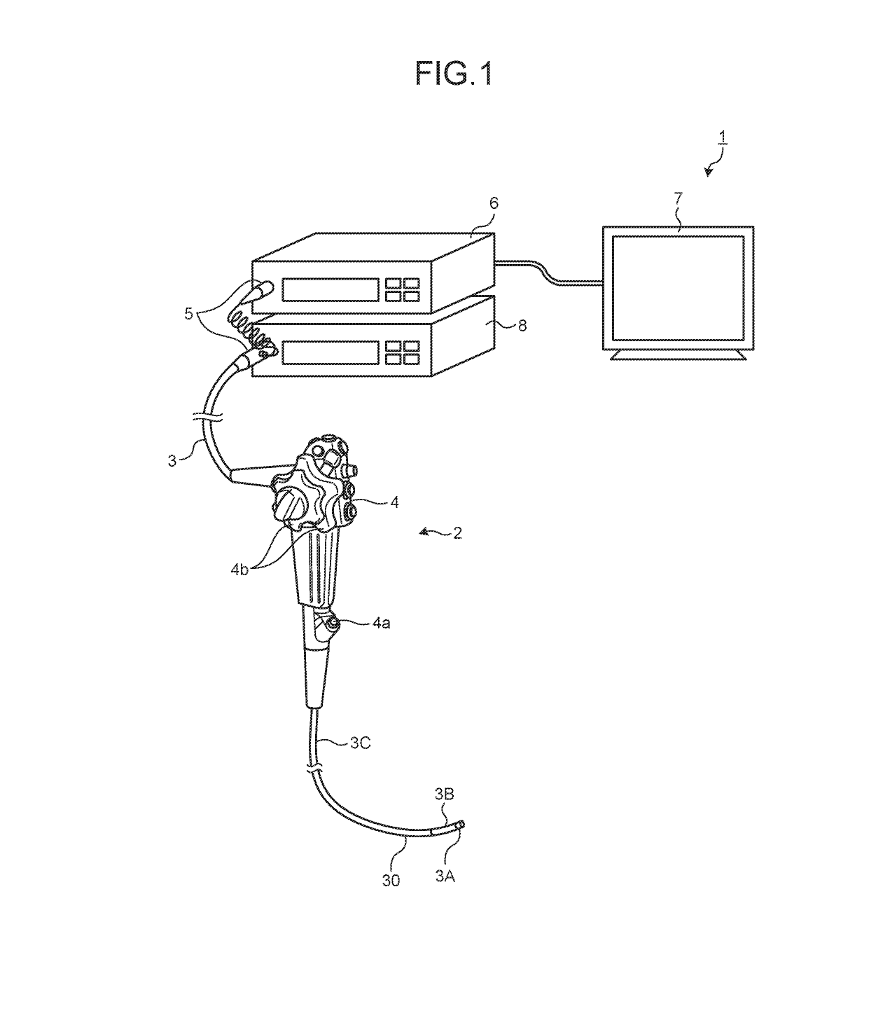

FIG. 1 is a diagram schematically illustrating an overall configuration of an endoscope system according to a first embodiment. An endoscope system 1 illustrated in FIG. 1 includes an endoscope 2, a universal cord 3 (transmission cable), a connector portion 5, a processor 6 (control device), a display device 7, and a light source device 8.

The endoscope 2 captures an in-vivo image of a subject and outputs an image signal (image data) to the processor 6 by inserting an insertion portion 30 into the subject. A bundle of electric cables inside the universal cord 3 extends to the insertion portion 30 of the endoscope 2 and is connected to the imaging device provided at a distal end portion 3A of the insertion portion 30. An operating unit 4 provided with various buttons and knobs for operating an endoscope function is connected to a proximal end side of the insertion portion 30 of the endoscope 2. The operating unit 4 has a treatment instrument insertion port 4a for inserting a treatment instrument such as a biological forceps, an electric scalpel, and a test probe in a body cavity of the subject.

The connector portion 5 is provided at a proximal end of the universal cord 3, and is connected to the processor 6 and the light source device 8. The connector portion 5 performs predetermined signal processing on the image signal output from the imaging device of the distal end portion 3A connected to the universal cord 3, performs A/D conversion on the image signal, and outputs a digital image signal to the processor 6.

The processor 6 performs predetermined image processing on the image signal output from the connector portion 5 and outputs the image signal to the display device 7. In addition, the processor 6 controls the entire endoscope system 1. The processor 6 is configured by use of a central processing unit (CPU) or the like.

The display device 7 displays an image corresponding to the image signal output from the processor 6. The display device 7 is configured by use of a display panel such as a liquid crystal display panel or an organic electro luminescence (EL) display panel, and the like.

The light source device 8 irradiates an object with illumination light from the distal end of the insertion portion 30 of the endoscope 2 via the connector portion 5 and the universal cord 3. The light source device 8 is configured by use of a xenon lamp, a light emitting diode (LED) lamp, or the like.

The insertion portion 30 includes the distal end portion 3A provided with the imaging device, a bending portion 3B which is connected to a proximal end side of the distal end portion 3A and freely bendable in a plurality of directions, and a flexible tube portion 3C connected to a proximal end side of the bending portion 3B. The image signal captured by the imaging device provided at the distal end portion 3A is connected to the connector portion 5 via the operating unit 4 by the universal cord 3 having a length of several meters, for example. The bending portion 3B is bent by operation of a bending operation knob 4b provided on the operating unit 4, and is freely bendable, for example in four directions of upward, downward, rightward, and leftward, in accordance with towing and relaxing of bending wire inserted into the insertion portion 30.

In addition, the endoscope 2 is provided with a light guide (not illustrated) for propagating the illumination light from the light source device 8, and is provided with an illumination lens (not illustrated) at an exit end of the illumination light by the light guide. The illumination lens is provided at the distal end portion 3A of the insertion portion 30.

Configuration of Distal End Portion of Endoscope

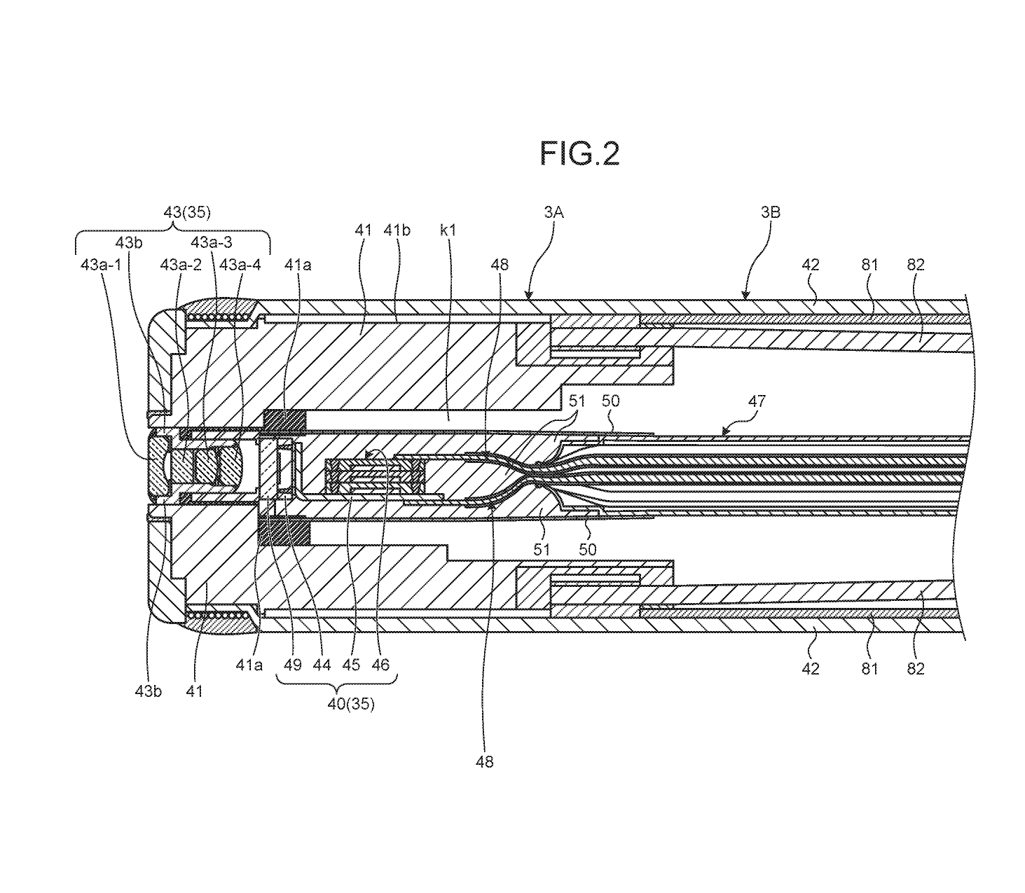

Next, a configuration of the distal end portion 3A of the endoscope 2 will be described in detail. FIG. 2 is a partial cross-sectional view of the distal end portion 3A of the endoscope 2, in a case where the distal end portion 3A is cut at a plane which is orthogonal to a substrate surface of the imaging device provided at the distal end portion 3A of the endoscope 2, and is parallel to an optical axis direction of the imaging device. In addition, FIG. 2 illustrates a part of the distal end portion 3A and the bending portion 3B of the insertion portion 30 of the endoscope 2.

As illustrated in FIG. 2, the bending portion 3B is freely bendable in four directions of upward, downward, rightward, and leftward, in accordance with towing and relaxing of bending wire 82 inserted into a bending tube 81 provided inside a cladding tube 42 described later. An imaging device 35 is provided inside the distal end portion 3A which extends from a distal end side of the bending portion 3B.

The imaging device 35 includes a lens unit 43 and an imaging unit 40 disposed on a proximal end side of the lens unit 43. The imaging device 35 is adhered to the inside of a distal end portion main body 41 by an adhesive 41a. The distal end portion main body 41 is formed into a cylindrical shape by a hard member or the like for forming an internal space k1 that accommodates the imaging device 35. A proximal end side outer peripheral portion 41b of the distal end portion main body 41 is covered with the flexible cladding tube 42. Members closer to the proximal end side than the distal end portion main body 41 are formed of flexible members such that the bending portion 3B is bendable. The distal end portion 3A where the distal end portion main body 41 is disposed becomes a hard portion of the insertion portion 30.

The lens unit 43 includes a plurality of objective lenses 43a-1 to 43a-4 and a lens holder 43b that holds the plurality of objective lenses 43a-1 to 43a-4. The lens unit 43 is fixed to the distal end portion main body 41 by insertion and fixation of a distal end of the lens holder 43b inside the distal end portion main body 41. The plurality of objective lenses 43a-1 to 43a-4 forms an object image.

The imaging unit 40 includes an image sensor 44 such as a charge coupled device (CCD) or a complementary metal oxide semiconductor (CMOS), the image sensor 44 having a light receiving unit that generates an electric signal (image signal) by performing photoelectric conversion by receiving light, a flexible printed board 45 (hereinafter referred to as a “FPC substrate 45”) that extends in the optical axis direction from a back side of a light receiving surface of the image sensor 44, a relay member 46 (interposer) made of a silicon substrate which is laminated on a surface of the FPC substrate 45 and on which a planar type electronic device is formed, the relay member 46 relaying the image sensor 44 and each signal cable 48 of an electric cable bundle 47, and a glass slid 49 that adheres to the image sensor 44 while covering the light receiving surface of the image sensor 44. The detailed configuration of the imaging unit 40 will be described later.

A proximal end of the signal cable 48 extends in a proximal end direction of the insertion portion 30. The electric cable bundle 47 is disposed in the distal end portion main body 41 so as to be insertable into the insertion portion 30, and extends to the connector portion 5 via the operating unit 4 and the universal cord 3 illustrated in FIG. 1.

The object image formed by the objective lenses 43a-1 to 43a-4 of the lens unit 43 is received and photoelectrically converted by the image sensor 44 disposed at an image formation position of the objective lenses 43a-1 to 43a-4, and converted into an image signal. The image signal generated by the image sensor 44 is output to the processor 6 via the FPC substrate 45, the relay member 46, the signal cable 48 connected to the relay member 46, and the connector portion 5.

An outer periphery of the imaging device 35 and an outer periphery of a distal end portion of the electric cable bundle 47 are covered with a heat shrinkable tube 50 in order to improve resistance. In the interior of the heat shrinkable tube 50, gaps among parts are filled with a sealing resin 51.

Detailed Configuration of Imaging Unit

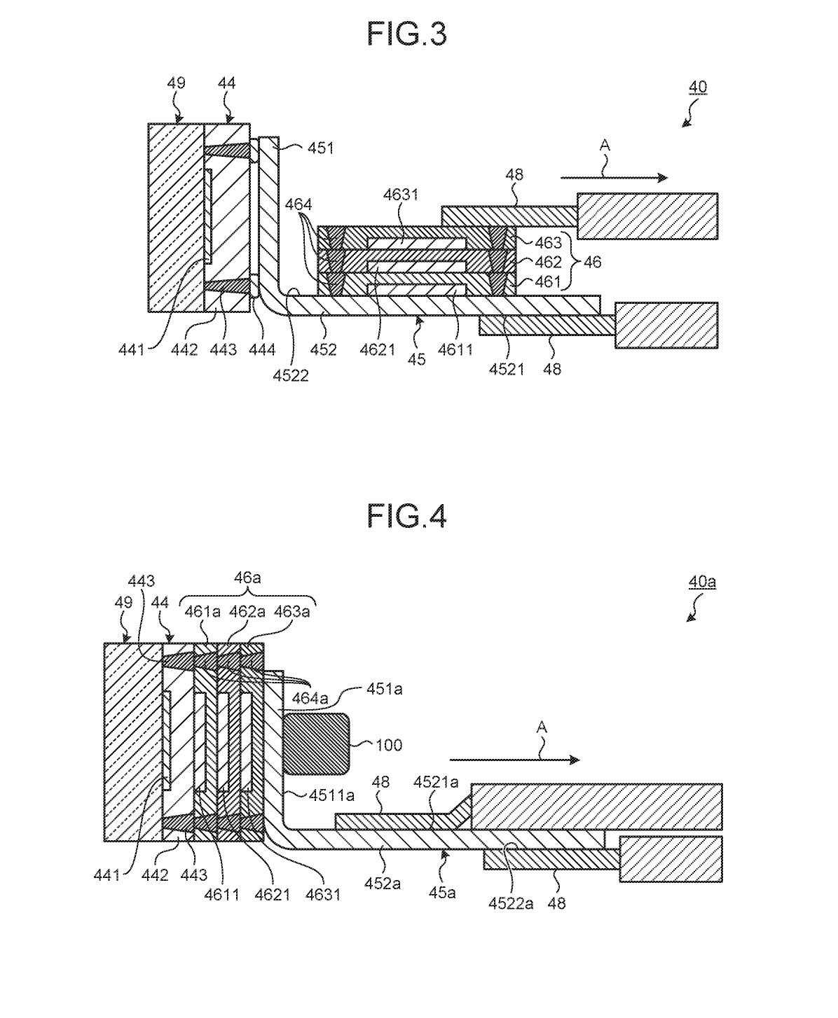

Next, the imaging unit 40 will be described in detail. FIG. 3 is a cross-sectional view of the imaging unit 40. The imaging unit 40 illustrated in FIG. 3 includes the above-mentioned glass lid 49, the image sensor 44, the FPC substrate 45, and the relay member 46.

The image sensor 44 includes a light receiving unit 441 that receives an object image formed by the objective lenses 43a-1 to 43a-4 of the lens unit 43 and performs photoelectric conversion to generate an image signal, a semiconductor substrate 442 on which the light receiving unit 441 is formed, a through via 443 (TSV: Through-Silicon Via) which is provided in the semiconductor substrate 442 and propagates the image signal generated by the light receiving unit 441, and a bump 444 that connects the through via 443 and the FPC substrate 45.

The FPC substrate 45 includes a first substrate 451 connected to a back side of the image sensor 44 via the bump 444, and a second substrate 452 that is continuously extended from one end of the first substrate 451 in a proximal end direction (an extending direction of the signal cable 48) and bent, the proximal end direction being orthogonal to the first substrate 451. In addition, the signal cable 48 is connected to a back surface 4521 of the second substrate 452.

The relay member 46 is provided on a back surface side of the image sensor 44 opposite to the light receiving unit 441 of the image sensor 44, and the relay member 46 includes a plurality of silicon substrates 461 to 463 (semiconductor layers) laminated on a surface 4522 of the second substrate 452 of the FPC substrate 45. The plurality of silicon substrates 461 to 463 has a plurality of electronic devices 4611, 4621, and 4631 formed with a planar type, respectively. The plurality of electronic devices 4611, 4621, and 4631 is laminated in a direction orthogonal (vertical direction) to the extending direction of the signal cable 48 (see arrow A). Each of the electronic devices 4611, 4621, and 4631 is connected by at least an adjacent silicon substrate, in particular via through vias 464 that pass through each layer. The plurality of electronic devices 4611, 4621, and 4631 is any one of a buffer, a capacitor, an inductor, and a resistor that amplify the image signal generated by the image sensor 44 and output the amplified image signal to the signal cable 48. The signal cable 48 is connected to an upper surface of the silicon substrate 463.

According to the first embodiment described above, the relay member 46 is provided on the back surface side of the image sensor 44 with respect to the light receiving unit 441, includes the silicon substrates 461 to 463 in which the plurality of electronic devices 4611, 4621, and 4631 is formed with a planar type, and relays the image sensor 44 and the signal cable 48. With this configuration, further miniaturization of the imaging unit 40 may be realized.

In addition, according to the first embodiment, further miniaturization of the imaging unit 40 may be realized by lamination of the plurality of silicon substrates 461 to 463 by the relay member 46.

Further, according to the first embodiment, the plurality of silicon substrates 461 to 463 is laminated in a direction parallel to the extending direction of the signal cable 48, and the signal cable 48 is connected to the relay member 46. With this configuration, further miniaturization of the imaging unit 40 may be realized.

Furthermore, according to the first embodiment, the plurality of silicon substrates 461 to 463 is laminated on the FPC substrate 45, and the signal cable 48 is connected to the back side of the FPC substrate 45. This configuration enables a design with flexibility.

Further, according to the first embodiment, since the plurality of silicon substrates 461 to 463 is provided in the internal space of the distal end portion main body 41 of the insertion portion 30 in the endoscope 2, miniaturization of the distal end portion 3A of the endoscope 2 may be realized.

### Second Embodiment

Next, a second embodiment will be described. The present second embodiment differs from the above-mentioned first embodiment only in the imaging unit 40 according to the first embodiment. Hereinafter, an imaging unit according to the present second embodiment will be described. Note that the same configurations as those of the endoscope system 1 according to the above-mentioned first embodiment are denoted by the same reference numerals, and the description thereof is omitted.

Detailed Configuration of Imaging Unit

FIG. 4 is a cross-sectional view of the imaging unit according to the second embodiment. An imaging unit 40a illustrated in FIG. 4 includes the glass lid 49, the image sensor 44, an FPC substrate 45a, a relay member 46a, and a passive element 100.

The relay member 46a includes a plurality of silicon substrates 461a to 463a laminated on the back surface side of the image sensor 44 opposite to the light receiving unit 441 of the image sensor 44. The plurality of silicon substrates 461a to 463a includes the plurality of electronic devices 4611, 4621, and 4631 formed with a planar type, respectively. The plurality of silicon substrates 461a to 463a is laminated on the back surface side of the image sensor 44 such that each area of the plurality of silicon substrates 461a to 463a is equal to or smaller than a projected area when the image sensor 44 is projected in the extending direction of the signal cable 48. The plurality of silicon substrates 461a to 463a is laminated in a direction parallel to the extending direction of the signal cable 48 (see arrow A). Each of the plurality of silicon substrates 461a to 463a is connected by through vias 464a that pass through each layer.

The FPC substrate 45a includes a first substrate 451aconnected to a back side of the relay member 46a via a bump (not illustrated), and a second substrate 452a that is continuously extended from one end of the first substrate 451a in a proximal end direction (the extending direction of the signal cable 48) and bent, the proximal end direction being orthogonal to the first substrate 451a. The signal cable 48 is connected to each of both surfaces 4521a and 4522a of the second substrate 452a of the FPC substrate 45a.

The passive element 100 is connected to a back surface 4511a side of the first substrate 451a of the FPC substrate 45a. The passive element 100 is at least one of a chip capacitor, an inductor, and a resistor.

According to the second embodiment as described above, the plurality of silicon substrates 461a to 463a is laminated in the direction parallel to the extending direction of the signal cable 48 (see arrow A). With this configuration, further miniaturization of the imaging unit 40a may be realized.

Furthermore, according to the second embodiment, the plurality of silicon substrates 461a to 463a is laminated on the back surface side of the image sensor 44 such that each area of the plurality of silicon substrates 461a to 463a is equal to or smaller than the projected area of the image sensor 44. With this configuration, further miniaturization of the imaging unit 40a may be realized.

### First Modification

Next, a first modification of the embodiments will be described. The present first modification of the embodiments differs from the above-mentioned first embodiment only in a configuration of the relay member 46 according to the first embodiment. Specifically, a relay member according to the present first modification of the embodiments forms planar type electronic devices on both surfaces of each of the plurality of laminated silicon substrates. Hereinafter, a configuration of the relay member according to the present first modification of the embodiments will be described.

FIG. 5 is a cross-sectional view of the relay member according to the first modification of the embodiments. A relay member 46b illustrated in FIG. 5 is formed by lamination of a plurality of silicon substrates 461b, 462b, and 463b. Planar type electronic devices 4611b, 4612b, 4621b, 4622b, 4631b, and 4632b are formed on both surfaces of the plurality of silicon substrates 461b, 462b, and 463b, respectively. Further, the plurality of silicon substrates 461b, 462b, and 463b is electrically connected to each other by through vias 464b and bumps 465a and 465b. The bump 465b may be disposed at a position different from a vertical direction of the through vias 464b to connect the silicon substrates 461b, 462b, and 463b, or may be disposed at the same position as the vertical direction of the through vias 464b to connect the silicon substrates 461b, 462b, and 463b. A resin layer (not illustrated) may be formed in each gap among the plurality of silicon substrates 461b, 462b, and 463b to reinforce connection strength among the silicon substrates. In addition, the electronic devices 4611b, 4612b, 4621b, 4622b, 4631b, and 4632b are any one of buffers, capacitors, inductors, and resistors that amplify the image signal generated by the image sensor 44 and output the amplified image signal to the signal cable 48.

According to the first modification of the embodiments as described above, by formation of the planar type electronic devices 4611b, 4612b, 4621b, 4622b, 4631b, and 4632b on both surfaces of the silicon substrates 461b, 462b, and 463b, further miniaturization may be achieved.

Note that, in the first modification of the embodiments, the planar type electronic devices 4611b, 4612b, 4621b, 4622b, 4631b, and 4632b are formed on both surfaces of the silicon substrates 461b, 462b, and 463b, respectively. However, for example, the plurality of planar type electronic devices may be formed in parallel on one surface of the silicon substrate 462b.

### Second Modification

Next, a second modification of the embodiments will be described. The present second modification of the embodiments differs from the above-mentioned first and second embodiments only in a configuration of the relay member according to the first and second embodiments. Specifically, a relay member according to the present second modification of the embodiments is formed by further lamination of a multilayer wiring layer on the laminated silicon substrates. Hereinafter, the configuration of the relay member according to the present second modification of the embodiments will be described.

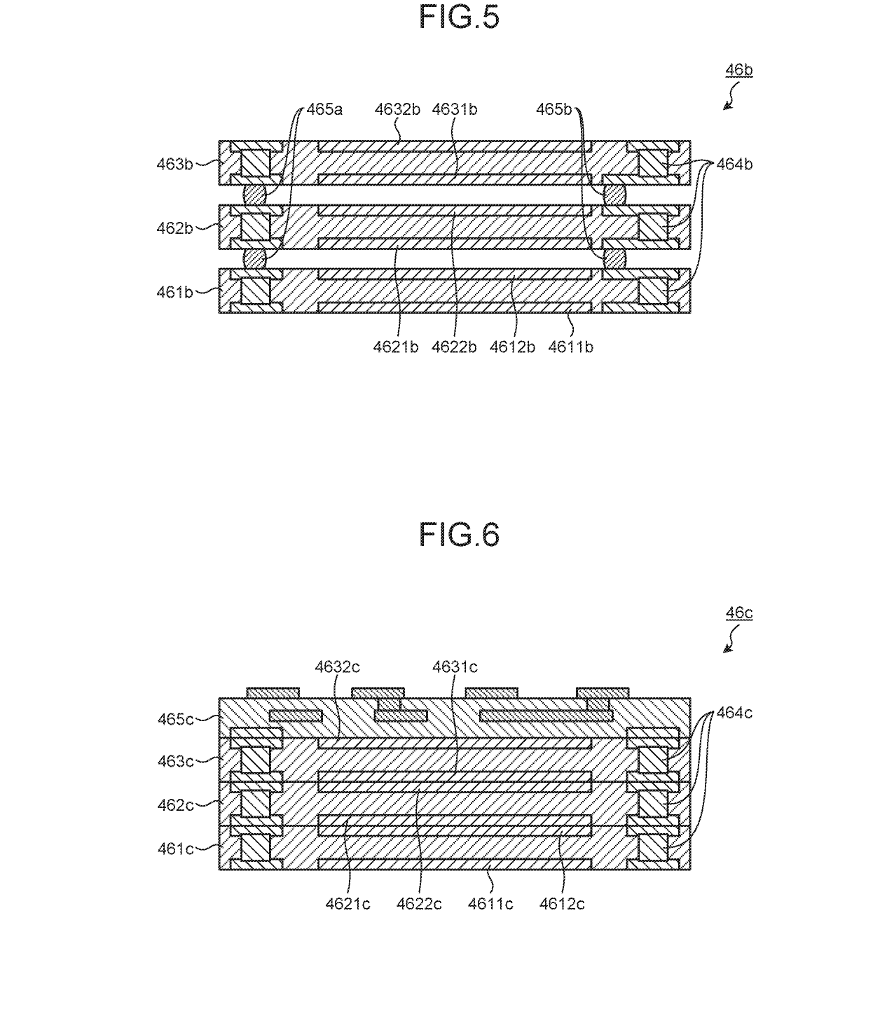

FIG. 6 is a cross-sectional view of the relay member according to the second modification of the embodiments. A relay member 46c illustrated in FIG. 6 is formed by lamination of a plurality of silicon substrates 461c, 462c, and 463c. Furthermore, a multilayer wiring layer 465c is laminated and formed on an outermost layer of the silicon substrate 463c. Planar type electronic devices 4611c, 4612c, 4621c, 4622c, 4631c, and 4632c are formed on both surfaces of the plurality of silicon substrates 461c, 462c, and 463c, respectively. Furthermore, each of the plurality of silicon substrates 461c, 462c, and 463c and the multilayer wiring layer 465c is electrically connected by through vias 464c. In the present modification, each of the planar type electronic devices 4611c, 4612c, 4621c, 4622c, 4631c, and 4632c is connected by directly bonding the through vias 464c, without use of a bump. The electronic devices 4611c, 4612c, 4621c, 4622c, 4631c, and 4632c are any one of buffers, capacitors, inductors, and resistors that amplify the image signal generated by the image sensor 44 and output the amplified image signal to the signal cable 48.

A material capable of connecting an electronic component or a signal cable by soldering, for example, an electrode in which an Au plating layer is formed on a Cu layer via an Ni barrier layer, is formed on an outermost surface of the multilayer wiring layer 465c. With this configuration, another electronic component, passive element, and signal cable 48 may be connected by soldering. Note that a multilayer FPC substrate may be laminated as the multilayer wiring layer 465c, or the multilayer wiring layer 465c may be formed on the silicon substrate 463c by a well-known build-up method.

According to the second modification of the embodiments described above, by lamination and formation of the multilayer wiring layer 465c on the outermost layer of the silicon substrate 463c, high-density wiring may be performed.

In addition, according to the second modification of the embodiments, by lamination and formation of the multilayer wiring layer 465c on the outermost layer of the silicon substrate 463c, another electronic component, passive element, and signal cable 48 may be connected by soldering.

Furthermore, according to the second modification of the embodiments, the planar type electronic devices 4611c, 4612c, 4621c, 4622c, 4631c, and 4632c are formed on both surfaces of the plurality of silicon substrates 461c, 462c, and 463c. With this configuration, further miniaturization may be achieved.

### Third Modification

Next, a third modification of the embodiments will be described. The present third modification of the embodiments differs from the above-mentioned first embodiment only in a configuration of the imaging unit 40 according to the first embodiment. Specifically, an imaging unit according to the present third modification of the embodiments is configured by use of an image sensor (imager chip) of a front illumination type (Front Side Illumination), and a relay unit is laminated on a back surface of the image sensor. Hereinafter, a configuration of the imaging unit according to the present third modification of the embodiments will be described.

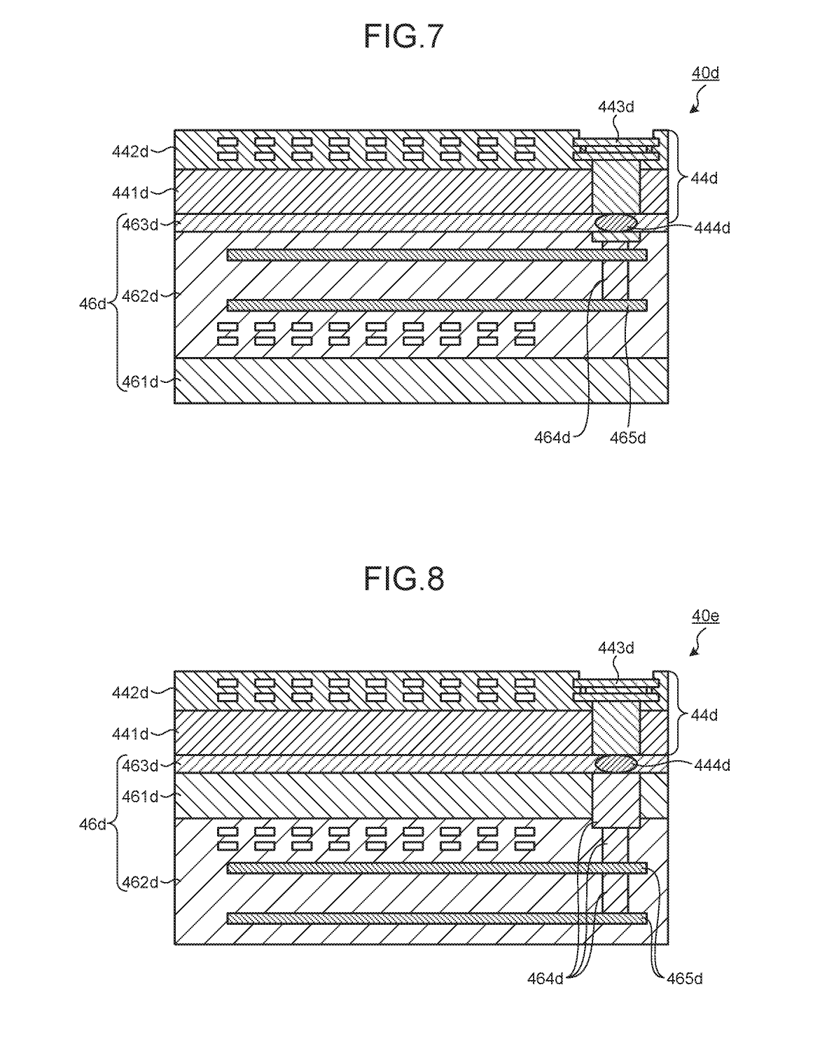

FIG. 7 is a schematic cross-sectional view of the imaging unit according to the third modification of the embodiments. An imaging unit 40d illustrated in FIG. 7 includes an image sensor 44d that generates an image signal (electric signal) by receiving light and performing photoelectric conversion, and a relay member 46d that relays the image sensor 44d and the signal cable 48.

The image sensor 44d includes a semiconductor substrate 441d on which a light receiving unit (pixel unit) in which a plurality of pixels (photodiodes) is arrayed in a two-dimensional matrix is formed, the light receiving unit outputting an electric signal by receiving light and performing photoelectric conversion, a wiring layer 442d laminated on the semiconductor substrate 441d, and a through via 464d.

The relay member 46d includes a semiconductor substrate 461d (silicon substrate) on which a circuit and the like are formed, an electronic device layer 462d formed by lamination of a dielectric and the like on the semiconductor substrate 461d, and a connecting portion 463d provided on an outermost layer of the electronic device layer 462d and connected to the image sensor 44d. The electronic device layer 462d is either a buffer that amplifies and outputs the image signal output from the image sensor 44d, or a bypass capacitor that flows an AC component such as noise to the ground. The electronic device layer 462d includes electrodes 465d. The electrodes 465d are electrically connected to a through via 443d via the through via 464d and a bump 444d.

According to the third modification of the embodiments described above, the relay member 46d is provided on the back surface side of the image sensor 44d. With this configuration, further miniaturization may be achieved.

In addition, in the third modification of the embodiments, a back surface side of the semiconductor substrate 441d of the image sensor 44d and a back surface side of the semiconductor substrate 461d of the relay member 46d may be connected and laminated. As illustrated in FIG. 8, in an imaging unit 40e, the semiconductor substrate 461d is electrically connected to the semiconductor substrate 441d via the bump 444d and the through via 443d. With this configuration, by providing the relay member 46d on the back surface side of the image sensor 44d, further miniaturization may be achieved.

### Fourth Modification

Next, a fourth modification of the embodiments will be described. The present fourth modification of the embodiments differs from the above-mentioned first embodiment only in a configuration of the imaging unit 40 according to the first embodiment. Specifically, an imaging unit according to the present fourth modification of the embodiments is configured by use of an image sensor (imager chip) of a back illumination type (Back Side Illumination), and a relay unit is laminated on a back surface of the image sensor. Hereinafter, a configuration of the imaging unit according to the present fourth modification of the embodiments will be described.

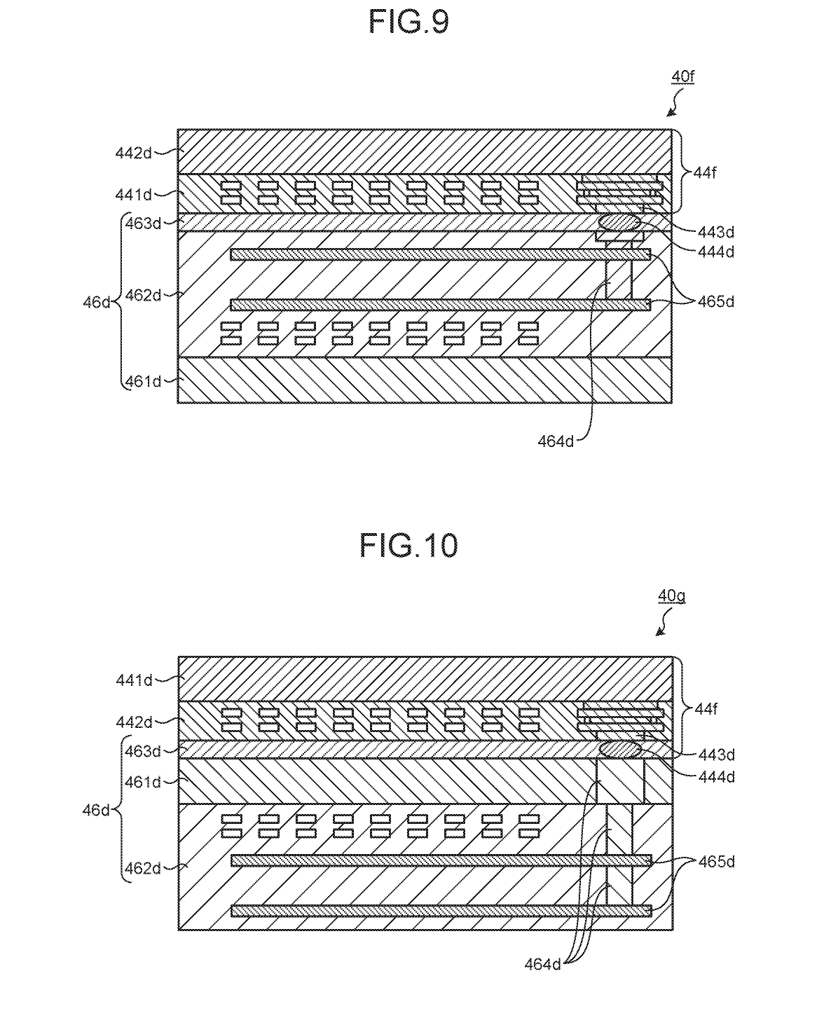

FIG. 9 is a schematic cross-sectional view of the imaging unit according to the fourth modification of the embodiments. An imaging unit 40f illustrated in FIG. 9 includes an image sensor 44f that generates an image signal (electric signal) by receiving light and performing photoelectric conversion, and the relay member 46d according to the above-mentioned third modification of the embodiment.

The image sensor 44f includes the semiconductor substrate 441d on which the light receiving unit (pixel unit) in which the plurality of pixels (photodiodes) is arrayed in the two-dimensional matrix is formed, the light receiving unit outputting the electric signal by receiving light and performing photoelectric conversion, the wiring layer 442d laminated on the semiconductor substrate 441d, and the through via 443d. The image sensor 44f is electrically connected to the relay member 46d via the through via 443d and the bump 444d.

According to the fourth modification of the embodiments described above, the relay member 46d is laminated on the back surface of the image sensor 44d. With this configuration, further miniaturization of the imaging unit 40f may be achieved.

In addition, in the fourth modification of the embodiments, a front surface side of a light receiving unit 442d of the image sensor 44f and the back surface side of the semiconductor substrate 461d of the relay member 46d may be connected and laminated. As illustrated in FIG. 10, in an imaging unit 40g, the front surface side of the light receiving unit 442d (wiring layer) of the image sensor 44f and the back surface side of the semiconductor substrate 461d of the relay member 46d are electrically connected via the bump 444d. With this configuration, further miniaturization may be achieved.

As described above, the present disclosure may include various embodiments not described here, and it is possible to make various design changes and the like within the scope of the technical idea specified by the claims.

According to the present disclosure, an effect of realizing further miniaturization may be achieved.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An imaging unit, comprising:
an image sensor configured to generate an image signal by receiving light and performing photoelectric conversion; and
a relay member including a plurality of silicon substrates laminated on a back surface side of the image sensor opposite to a light receiving surface of the image sensor, planar type electronic devices being formed on the silicon substrates, and relay the image sensor and a signal cable that transmits the image signal,
wherein the relay member includes a multilayer wiring layer laminated on an outermost surface of the silicon substrate, and
wherein the multilayer wiring layer includes, on an outermost surface, a material allowing the signal cable to be connected.

2. The imaging unit according to claim 1, wherein the plurality of silicon substrates are laminated in a direction orthogonal to an extending direction of the signal cable.

3. The imaging unit according to claim 1, wherein
the relay member further includes a flexible printed board that extends in parallel to an extending direction of the signal cable, and
the plurality of silicon substrates are laminated on the flexible printed board.

4. The imaging unit according to claim 3, wherein the multilayer wiring layer is formed of flexible printed boards.

5. The imaging unit according to claim 1, wherein the plurality of silicon substrates are laminated in a direction parallel to an extending direction of the signal cable.

6. The imaging unit according to claim 5, wherein an area of each of the plurality of silicon substrates is equal to or less than a projected area when the image sensor is projected in the extending direction of the signal cable.

7. The imaging unit according to claim 1, wherein the plurality of silicon substrates are connected by a through via that passes through at least an adjacent silicon substrate.

8. The imaging unit according to claim 1, wherein the electronic device is formed on each one of both surfaces of each of the plurality of silicon substrates.

9. The imaging unit according to claim 1, wherein the electronic device is at least any one of a buffer, a capacitor, an inductor and a resistor.

10. An endoscope, comprising:
the imaging unit according to claim 1; and
an insertion portion that includes a cylindrical distal end portion formed of a hard member and is insertable into a subject,
